(19) United States
(12) Patent Application Publication
(10) Pub. No.: US 2007/0048782 A1
(43) Pub. Date: Mar. 1, 2007

(54) Methods for assessing and treating leukemia

(76) Inventor: Mitch Raponi, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/589,660

(22) Filed: Oct. 30, 2006

Related U.S. Application Data
(62) Division of application No. 10/283,975, filed on Oct. 30, 2002.
(60) Provisional application No. 60/340,938, filed on Oct. 30, 2001.
(60) Provisional application No. 60/338,997, filed on Oct. 30, 2001.
(60) Provisional application No. 60/340,081, filed on Oct. 30, 2001.
(60) Provisional application No. 60/341,012, filed on Oct. 30, 2001.

(51) Int. Cl. C12Q 1/68

(52) U.S. Cl. 435/6

(57) ABSTRACT

Methods for treating leukemia patients include analyzing gene expression profiles of a patient to determine whether the patient is likely to respond to treatment with farnesyl transferase inhibitor (FTI) and, optionally, other therapeutics. The methods are also useful for monitoring patient therapy and for selecting a course of therapy. Genes modulated in response to FTI treatment are provided and are used in formulating the profiles.

This application claims the benefit of the following US applications: US National application Ser. No. 10/283,975 filed Oct. 30, 2002; US Provisional applications 60/340,938 filed Oct. 30, 2001; 60/338,997 filed Oct. 30, 2001; 60/340,081 filed Oct. 30, 2001, and 60/341,012 filed Oct. 30, 2001. This invention relates to diagnostics, prognostics, and treatments for leukemia based on the gene expression profiles of leukemia cells.

### BACKGROUND

Some molecules, such as Ras, that are implicated in cancers must be farnesylated by the farnesyl transferase enzyme in order to interact with the inner leaflet of the plasma membrane of the cell and become involved in various signaling pathways. Ras is not the only protein implicated in cancer that has a CAAX box that is prenylated. Farnesyl transferase inhibitors (FTIs) are therapeutic agents that inhibit the covalent attachment of the carbon farnesyl moieties to the C-terminal CAAX motif of various proteins. They have utility in the treatment of cancers and proliferative disorders such as leukemia. Acute myelogenous leukemia (AML) is among the diseases that can most beneficially be addressed with FTIs.

As is true in the case of many treatment regimens, some patients respond to treatment with FTIs and others do not. Prescribing the treatment to a patient who is unlikely to respond to it is not desirable. Thus, it would be useful to know how a patient could be expected to respond to such treatment before a drug is administered so that non-responders would not be unnecessarily treated and so that those with the best chance of benefiting from the drug are properly treated and monitored. Further, of those who respond to treatment, there may be varying degrees of response. Treatment with therapeutics other than FTIs or treatment with therapeutics in addition to FTIs may be beneficial for those patients who would not respond to FTIs or in whom response to FTIs alone is less than desired.

### SUMMARY OF THE INVENTION

The invention is a method of treating a patient with leukemia with an FTI. In one such method, the patient's gene expression profile is analyzed to determine whether the patient is likely to respond to the FTI and treating a patient with the FTI if they are likely to respond.

In another aspect of the invention, a patient with leukemia is monitored for treatment with an FTI in which the patient's gene expression profile is analyzed to determine whether the patient is responding to the FTI and treating a patient with the FTI if they are likely to respond in a desirable fashion.

In yet another aspect of the invention, a patient is treated if the gene expression profile shows up regulation of one or more particular genes indicative of FTI responders.

In yet another aspect of the invention, gene expression profiles indicative of FTI responders are those which show at least a 1.5, 1.7, or 2 fold difference relative to FTI non-responders.

In yet another aspect of the invention, a patient is treated if the gene expression profile shows down regulation of one or more particular genes indicative of FTI responders

In yet another aspect of the invention, a patient is treated if the gene expression profile shows modulation of a gene selected from the group of genes identified in Tables 1-3 infra.

In yet another aspect of the invention, the FTI is a quinilone or quinoline derivative.

In yet another aspect of the invention, the FTI is (B)-6-[amino(4-chlorophenyl)(1-methyl-1H-imidazol-5-yl)methyl]-4-(3-chlorophenyl)-1-methyl-2(1H)-quinolinone).

Articles used in practicing the methods are also an aspect of the invention. Such articles include gene expression profiles or representations of them that are fixed in computer readable media. Other articles according to the invention include nucleic acid arrays used to determine the gene expression profiles of the invention.

In another aspect of the invention, a method of treating a patient with leukemia comprises administering an FTI and a therapeutic composition that modulates the MAPK/ERK signaling pathways, TGFβ, WNT or apoptotic pathways.

In another aspect of the invention, the patient is treated with an FTI and a therapeutic composition selected from the group consisting of tyrosine kinase inhibitors, MEK kinase inhibitors, PI3K kinase inhibitors, MAP kinase inhibitors, apoptosis modulators and combinations thereof.

In yet another aspect of this invention, the gene expression profile of a patient with leukemia is analyzed to determine whether the patient is likely to respond to an FTI or if the patient would likely benefit from the combination of an FTI and another drug. The patient is then treated with such combination or, if the patient is unlikely to respond to an FTI, the patient is treated with drug selected from the group consisting of tyrosine kinase inhibitors, MEK kinase inhibitors, PI3K kinase inhibitors, MAP kinase inhibitors, apoptosis modulators and combinations thereof.

### BRIEF DESCRIPTION OF THE DRAWINGS

FIG. 1 is an example of a graphical display of gene expression patterns used to analyze the gene expression profiles of this invention.

FIG. 2 is a schematic diagram of the MAPK/ERK pathway.

FIG. 3 is a schematic diagram of the TGFβ and Wnt pathway.

FIG. 4 is a schematic diagram of the apoptotic pathway.

### DETAILED DESCRIPTION

The therapeutic agents referred to in this specification are FTIs. They take on a multitude of forms but share the essential inhibitory function of interfering with or lessening the farnesylation of proteins implicated in cancer and proliferative diseases. Preferably, the FTIs are those indicated for the treatment of leukemias such as AML. A patient who responds to an FTI is one in whom a reduction of more than 50% of blast cells is seen in bone marrow following treatment with the FTI.

Numerous FTIs are within the scope of the invention and include those described in U.S. Pat. No. 5,976,851 to Brown et al; U.S. Pat. No. 5,972,984 to Anthony et al.; U.S. Pat. No. 5,972,966 to deSolms; U.S. Pat. No. 5,968,965 to Dinsmore et al.; U.S. Pat. No. 5,968,952 to Venet et al.; U.S. Pat. No. 6,187,786 to Venet et al.; U.S. Pat. No. 6,169,096 to Venet et al.; U.S. Pat. No. 6,037,350 to Venet et. al.; U.S. Pat. No. 6,177,432 to Angibaud et al.; U.S. Pat. No. 5,965,578 to Graham et al.; U.S. Pat. No. 5,965,539 to Sebti et al.; U.S. Pat. No. 5,958,939 to Afonso et al.; U.S. Pat. No. 5,939,557 to Anthony et al.; U.S. Pat. No. 5,936,097 to Commercon et al.; U.S. Pat. No. 5,891,889 to Anthony et al.; U.S. Pat. No. 5,889,053 to Baudin et al.; U.S. Pat. No. 5,880,140 to Anthony; U.S. Pat. No. 5,872,135 to deSolms; U.S. Pat. No. 5,869,682 to deSolms; U.S. Pat. No. 5,861,529 to Baudoin; U.S. Pat. No. 5,859,015 to Graham et al.; U.S. Pat. No. 5,856,439 to Clerc; U.S. Pat. No. 5,856,326 to Anthony et al.; U.S. Pat. No. 5,852,010 to Graham et al.; U.S. Pat. No. 5,843,941 to Marsters et al.; U.S. Pat. No. 5,807,852 to Doll; U.S. Pat. No. 5,780,492 to Dinsmore et al.; U.S. Pat. No. 5,773,455 to Dong et al.; U.S. Pat. No. 5,767,274 to Kim et al.; U.S. Pat. No. 5,756,528 to Anthony et al.; U.S. Pat. No. 5,750,567 to Baudoin et al.; U.S. Pat. No. 5,721,236 to Bishop et al,; U.S. Pat. No. 5,700,806 to Doll et al.; U.S. Pat. No. 5,661,161 to Anthony et al.; U.S. Pat. No. 5,602,098 to Sebti et al.; U.S. Pat. No. 5,585,359 to Breslin et al.; U.S. Pat. No. 5,578,629 to Ciccarone et al.; U.S. Pat. No. 5,534,537 to Ciccarone et al.; U.S. Pat. No. 5,532,359 to Marsters et al.; U.S. Pat. No. 5,523,430 to Patel et al.; U.S. Pat. No. 5,504,212 to deSolms et al.; U.S. Pat. No. 5,491,164 to deSolms et al.; U.S. Pat. No. 5,420,245 to Brown et al.; and U.S. Pat. No. 5,238,922 to Graham et al. each of which is incorporated herein by reference. Non-peptidal, so-called “small molecule” therapeutics are preferred. More preferred FTIs are quinolines or quinoline derivatives such as:
7-(3-chlorophenyl)-9-[(4-chlorophenyl)-1H-imidazol-1-ylmethyl]-2,3-dihydro-1H,5H-benzo[ij]quinolizin-5-one,
7-(3-chlorophenyl)-9-[(4-chlorophenyl)-1H-imidazol-1-ylmethyl]-1,2-dihydro-4H-pyrrolo[3,2,1-ij]quinoline-4-one,
8-[amino(4-chlorophenyl)(1-methyl-1H-imidazol-5-yl),methyl]-6-(3-chlorophenyl)-1,2-dihydro-4H-pyrrolo[3,2,1-ij]quinolin-4-one, and
8-[amino(4-chlorophenyl)(1-methyl-1H-imidazol-5-yl)methyl]-6-(3-chlorophenyl)-2,3-dihydro-1H,5H-benzo[ij]quinolizin-5-one.
   The most preferred FTI is (B)-6-[amino(4-chlorophenyl)(1-methyl-1H-imidazol-5-yl)methyl]-4-(3-chlorophenyl)-1-methyl-2(1H)-quinolinone).

In the aspect of the invention comprising treating leukemia with FTIs and other therapeutic agents, The therapeutic agents referred to in this specification are those that have an effect on the biological pathway explicated through the gene expression analysis of leukemic cells subjected to treatment with quinilone-based FTIs.

The mere presence of nucleic acid sequences having the potential to express proteins or peptides (“genes”) within the genome is not determinative of whether a protein or peptide is expressed in a given cell. Whether or not a given gene capable of expressing proteins or peptides does so and to what extent such expression occurs, if at all, is determined by a variety of complex factors. Irrespective of difficulties in understanding and assessing these factors, assaying gene expression can provide useful information about the cellular response to a given stimulus such as the introduction of a drug or other therapeutic agent. Relative indications of the degree to which genes are active or inactive can be found in gene expression profiles. The gene expression profiles of this invention are used to identify and treat patients who will likely benefit from a given therapy or exclude patients from a given therapy where the patient likely would experience little or no beneficial response to the drug or therapy.

Preferred methods for establishing gene expression profiles (including those used to arrive at the explication of the relevant biological pathways) include determining the amount of RNA that is produced by a gene that can code for a protein or peptide. This is accomplished by reverse transcription PCR (RT-PCR), competitive RT-PCR, real time RT-PCR, differential display RT-PCR, Northern Blot analysis and other related tests. While it is possible to conduct these techniques using individual PCR reactions, it is best to amplify copy DNA (cDNA) or copy RNA (cRNA) produced from mRNA and analyze it via microarray. A number of different array configurations and methods for their production are known to those of skill in the art and are described in U.S. Patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; the disclosures of which are herein incorporated by reference.

Microarray technology allows for the measurement of the steady-state mRNA level of thousands of genes simultaneously thereby presenting a powerful tool for identifying the effect of FTIs on cell biology and the likely effect of treatment based on analysis of such effects. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. No. 6,271,002 to Linsley, et al.; U.S. Pat. No. 6,218,122 to Friend, et al.; U.S. Pat. No. 6,218,114 to Peck, et al.; and U.S. Pat. No. 6,004,755 to Wang, et al., the disclosure of each of which is incorporated herein by reference.

Analysis of the expression levels is conducted by comparing such intensities. This is best done by generating a ratio matrix of the expression intensities of genes in a test sample versus those in a control sample. For instance, the gene expression intensities from a tissue that has been treated with a drug can be compared with the expression intensities generated from the same tissue that has not been treated with the drug. A ratio of these expression intensities indicates the fold-change in gene expression between the test and control samples.

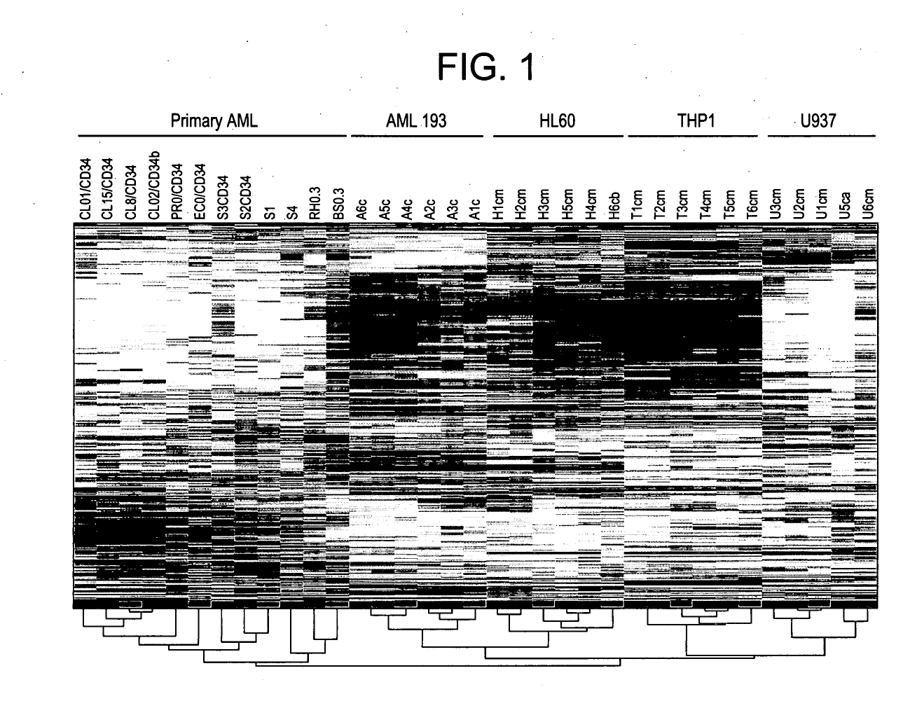

Gene expression profiles can also be displayed in a number of ways. The most common method is to arrange a ratio matrix into a graphical dendogram where columns indicate test samples and rows indicate genes. The data is arranged so genes that have similar expression profiles are proximal to each other (e.g., FIG. 1). The expression ratio for each gene is visualized as a color. For example, a ratio less than one (indicating down-regulation) may appear in the blue portion of the spectrum while a ratio greater than one (indicating up-regulation) may appear as a color in the red portion of the specrtum. Commercially available computer software programs are available to display such data including “OMNIVIZ PRO” software from Batelle and “TREE VIEW” software from Stanford

The genes that are differentially expressed are either up regulated or down regulated in diseased cells following treatment with an FTI. Up regulation and down regulation are relative terms meaning that a detectable difference (beyond the contribution of noise in the system used to measure it) is found in the amount of expression of the genes relative to some baseline. In this case, the baseline is the measured gene expression of the untreated diseased cell. The genes of interest in the treated diseased cells are then either up regulated or down regulated relative to the baseline level using the same measurement method. Preferably, levels of up and down regulation are distinguished based on fold changes of the intensity measurements of hybridized microarray probes. A 1.5 fold difference is preferred for making such distinctions. That is, before a gene is said to be differentially expressed in treated versus untreated diseased cells, the treated cell is found to yield at least 1.5 times more, or 1.5 times less intensity than the untreated cells. A 1.7 fold difference is more preferred and a 2 or more fold difference in gene expression measurement is most preferred. Table 3 lists genes that were commonly modulated across all cell lines and in responder samples.

A portfolio of genes is a set of genes grouped so that information obtained about them provides the basis for making a clinically relevant judgment such as a diagnosis, prognosis, or treatment choice. In this case, the judgments supported by the portfolios involve the treatment of leukemias with FTI's. Portfolios of gene expression profiles can be comprised of combinations of genes shown in Tables 1-3.

One method of the invention involves comparing gene expression profiles for various genes to determine whether a person is likely to respond to the use of a therapeutic agent. Having established the gene expression profiles that distinguish responder from nonresponder, the gene expression profiles of each are fixed in a medium such as a computer readable medium as described below. A patient sample is obtained that contains diseased cells (such as hematopoietic blast cells in the case of AML) is then obtained. Sample RNA is then obtained and amplified from the diseased patient cell and a gene expression profile is obtained, preferably via micro-array, for genes in the appropriate portfolios. The expression profiles of the samples are then compared to those previously determined as responder and non-responder. If the sample expression patterns are consistent with an FTI responder expression pattern then treatment with an FTI could be indicated (in the absence of countervailing medical considerations). If the sample expression patterns are consistent with an FTI non-responder expression pattern then treatment with an FTI would not be indicated. Preferably, consistency of expression patterns is determined based on intensity measurements of micro-array reading as described above.

In similar fashion, gene expression profile analysis can be conducted to monitor treatment response. In one aspect of this method, gene expression analysis as described above is conducted on a patient treated with an FTI at various periods throughout the course of treatment. If the gene expression patterns are consistent with a responder then the patient's therapy is continued. If it is not, then the patient's therapy is altered as with additional therapeutics such as tyrosine kinase inhibitor, changes to the dosage, or elimination of FTI treatment. Such analysis permits intervention and therapy adjustment prior to detectable clinical indicia or in the face of otherwise ambiguous clinical indicia.

It is possible to attain ambiguous results in which some gene expression profiles are recorded that are in some respects indicative of a responder and in other respects indicative of a non-responder. For example, the profiles may show that three genes are up-regulated consistent with a responder but that another gene is not up-regulated as would ordinarily be the case for a responder. In such a case, statistical algorithms can be applied to determine the probability that the patient will respond or not respond to the drug. Statistical algorithms suitable for this purpose are well known and are available.

Articles of this invention are representations of the gene expression profiles useful for treating, diagnosing, prognosticating, staging, and otherwise assessing diseases that are reduced to a medium that can be automatically read such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. FIG. 1 shows an example of the graphical display of such a recordation. Clustering algorithms such as those incorporated in “OMNIVIZ” and “TREE VIEW” computer programs mentioned above can best assist in the visualization of such data.

Additional articles according to the invention are nucleic acid arrays (e.g. cDNA or oligonucleotide arrays), as described above, configured to discern the gene expression profiles of the invention.

Using clustering analysis (including the algorithms mentioned above) one can compare the expression levels of patient samples to establish regulatory relationships among genes with a certain statistical confidence. A dynamic map was constructed based upon such expression data. Such a genetic network map is useful for drug discovery. For example, once basic genes of interest were identified, a list of potential up-stream regulatory genes was found using such a genetic network map. The genes so identified or their expression products were then analyzed for their use as drug targets. In some embodiments, the regulatory function of the particular genes identified was used to identify therapeutics for use in treating leukemia.

The regulation of transcription, RNA processing and RNA editing are all accomplished by proteins which are coded by their own genes. In addition, DNA sequences can exert long range control over the expression of other genes by positional effects. Therefore, the expression of genes is often regulated by the expression of other genes. Those regulatory genes are called upstream genes, relative to the regulated or down-stream genes. In a simple regulatory pathway:
A++>B−−>C++>D
where: A, B, C, D are genes
++ up-regulates
−− down-regulates
   Gene A is an up-stream gene of gene B and B is an up-stream gene of C. One of skill in the art would appreciate that the network is frequently looped and inter-connected. In some instances, the expression of a gene is regulated by its own product as either a positive or negative feedback.

Cluster analysis methods were used to group genes whose expression level is correlated. Methods for cluster analysis are described in detail in Harfigan (1975) Clustering Algorithms, NY, John Wile and Sons, Inc, and Everritt, (1980) Cluster Analysis 2nd. Ed. London Heineman Educational books, Ltd., incorporated herein for all purposed by reference. Path analysis was used to decompose relations among variables and for testing causal models for the genetic networks. Multiple primary targets of a drug in leukemic cells were identified as were drugs/drug classes useful in treating such cells. According to the current invention, drugs are any compounds of any degree of complexity that perturb a biological system.

The biological effect of a drug may be a consequence of drug-mediated changes in the rate of transcription or degradation of one or more species of RNA, the rate or extent of translation or post-translational processing of one or more polypeptides, the rate or extent of the degradation of one or more proteins, the inhibition or stimulation of the action or activity of one or more proteins, and so forth. In addition to the FTI's that are preferred, the preferred drugs of this invention are those that modulate the MAPK/ERK signaling pathways, TGFβ, WNT or apoptotic pathways. These include, without limitation, tyrosine kinase inhibitors, MEK kinase inhibitors, P13K kinase inhibitors, MAP kinase inhibitors, apoptosis modulators and combinations thereof. Exemplary drugs that are most preferred among these are the “GLEEVEC” tyrosine kinase inhibitor of Novartis, U-0126 MAP kinase inhibitor, PD-098059 MAP kinase inhibitor, SB-203580 MAP kinase inhibitor, and antisense, ribozyme, and DNAzyme Bcl-XL anti-apoptotics. Examples of other useful drugs include, without limitation, the calanolides of U.S. Pat. No. 6,306,897; the substituted bicyclics of U.S. Pat. No. 6,284,764; the indolines of U.S. Pat. No. 6,133,305; and the antisense oligonucleotides of U.S. Pat. No. 6,271,210.

As noted, the drugs of the instant invention can be therapeutics directed to gene therapy or antisense therapy. Oligonucleotides with sequences complementary to a mRNA sequence can be introduced into cells to block the translation of the mRNA, thus blocking the function of the gene encoding the mRNA. The use of oligonucleotides to block gene expression is described, for example, in, Strachan and Read, Human Molecular Genetics, 1996, incorporated herein by reference.

These antisense molecules may be DNA, stable derivatives of DNA such as phosphorothioates or methylphosphonates, RNA, stable derivatives of RNA such as 2′-O-alkylRNA, or other antisense oligonucleotide mimetics. Antisense molecules may be introduced into cells by microinjection, liposome encapsulation or by expression from vectors harboring the antisense sequence.

In the case of gene therapy, the gene of interest can be ligated into viral vectors that mediate transfer of the therapeutic DNA by infection of recipient host cells. Suitable viral vectors include retrovirus, adenovirus, adeno-associated virus, herpes virus, vaccinia virus, polio virus and the like. Alternatively, therapeutic DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo gene therapy. Protocols for molecular methodology of gene therapy suitable for use with the gene is described in Gene Therapy Protocols, edited by Paul D. Robbins, Human press, Totawa N.J., 1996.

Pharmaceutically useful compositions comprising the drugs of this invention may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the drug. The effective amount of the drug may vary according to a variety of factors such as the individual's condition, weight, sex and age. Other factors include the mode of administration. The pharmaceutical compositions may be provided to the individual by a variety of routes such as subcutaneous, topical, oral and intramuscular.

The drugs of this invention include chemical derivatives of the base molecules of the drug. That is, they may contain additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages defined by routine testing in order to obtain optimal inhibition or activity while minimizing any potential toxicity. In addition, co-administration or sequential administration of other agents may be desirable.

The drugs of this invention can be administered in a wide variety of therapeutic dosage forms in conventional vehicles for administration. For example, the drugs can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a modulating agent.

The daily dosage of the products may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages are adjusted when combined to achieve desired effects. On the other hand, dosages of these various agents may be independently optimized and combined to achieve a synergistic result wherein the pathology is reduced more than it would be if either agent were used alone.

Advantageously, compounds or modulators used in the present invention may be administered in a single daily dose, or the total daily dosage may be administered in divided doses of two, three or four times daily. Furthermore, compounds or modulators for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds or modulators in the present invention is selected in accordance with a variety of factors including type, species, age, weight, sex and medical condition of the patient; the severity of the condition to be treated; the route of administration; the renal and hepatic function of the patient; and the particular drug employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

The drugs of this invention can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations, which generally contain suitable preservatives, are employed when intravenous administration is desired.

The drugs in the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Drugs in the present invention may also be delivered by the use of monoclonal antibodies as individual carriers to which the compound molecules are coupled. The drugs in the present invention may also be coupled with soluble polymers as targetable drug carriers. Such polymers can include polyvinyl-pyrrolidone, pyran copolymer, polyhydroxypropylmethacryl-amidephenol, polyhydroxy-ethylaspartamidephenol, or polyethyl-eneoxidepolylysine substituted with palmitoyl residues. Furthermore, the drugs in the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For oral administration, the drugs may be administered in capsule, tablet, or bolus form or alternatively they can be mixed with feed. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the drugs and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated and the type and severity of the infection. The active ingredient may also be administered by simply mixing the compound with the feedstuff or by applying the compound to the surface of the foodstuff.

The compounds or modulators may alternatively be administered parenterally via injection of a formulation consisting of the active ingredient dissolved in an inert liquid carrier. Injection may be either intramuscular, intraruminal, intratracheal, or subcutaneous. The injectable formulation consists of the active ingredient mixed with an appropriate inert liquid carrier. Acceptable liquid carriers include the vegetable oils such as peanut oil, cotton seed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the active ingredient in the liquid carrier such that the final formulation contains from 0.005 to 10% by weight of the active ingredient.

The invention is further illustrated by the following nonlimiting examples.

### EXAMPLE 1

### Cell Culture

The AML-like cell lines HL-60 (promyelocytic) and U-937 (promonocytic) were obtained from the ATCC. AML-193 (monocytic) and THP-1 (monocytic) cells were obtained from the RW Johnson Pharmaceutical Research Center, San Diego. Cells were grown in Roswell Park Memorial Institute medium (RPMI) with 20% Fetal Bovine Serum (FBS). AML-193 was also supplemented with granulocyte-macrophage colony-stimulating factor (GM-CSF) (10 ng/ml), insulin (0.005 mg/ml), and transferrin (0.005 mg/ml).

### EXAMPLE 2

### Toxic Dose Assay

The cells of Example 1 were inoculated into 6-well plates at an initial concentration of 1×10⁵ cells/ml. (B)-6-[amino(4-chlorophenyl)(1-methyl-1H-imidazol-5-yl)methyl]-4-(3-chlorophenyl)-1-methyl-2(1H)-quinolinone) was added at concentrations ranging form 0.5 to 500 nM in 3 μl of DMSO directly to the culture medium. Control cells from Example 1 were grown in medium alone or in medium supplemented with vehicle (0.1% DMSO). Cell numbers were counted at days four and seven in a hemocytometer and cell viability was determined by trypan blue exclusion assay. The IC₅₀ was defined as the dose at which the number of viable cells in the treated sample was 50% of that in the control at day seven. Calculations were made based on duplicate runs of the experiment. The IC₅₀ of the four cell lines was calculated after seven days of treatment with the FTI. AML-193 had an IC₅₀ of 134 nM, HL-60 had an IC₅₀ of 24 nM, THP-1 had an IC₅₀ of 19 nM, and U-937 had an IC₅₀ of 44 nM. This indicated that the four AML-like cell lines were sensitive to FTI treatement.

### EXAMPLE 3

### Time Course Assay

Duplicate cultures of the cells of Example 1 were inoculated into 6-well plates at an initial concentration of 1×10⁵ cells/ml. (B)-6-[amino(4-chlorophenyl)(1-methyl-1H-imidazol-5-yl)methyl]-4-(3-chlorophenyl)-1-methyl-2(1H)-quinolinone) was supplemented at a concentration of 100 nM in 3 μl of DMSO directly to the culture medium. The concentration of 100 nM was chosen for the subsequent time course experiments to normalize the treatment protocol based, in part, on the results of Example 2. Duplicate control cultures were grown in medium containing 0.1% DMSO. Duplicate cultures were harvested daily for a total of six days. Cells were counted, assayed for viability, and total RNA isolated according to the manufacturer's protocol (Qiagen RNeasy). The analysis showed that cells from different cell lines were effected at different times. RNA was treated with DNase1 (Qiagen DNase1 kit) to remove any residual genomic DNA. Linear amplification of RNA was conducted according to the procedure described in U.S. Pat. No. 5,545,522 to Van Gelder et. al. Aliquots of 5 μg of aRNA were then prepared for hybridization to cDNA arrays.

### EXAMPLE 4

### Bone Marrow Processing

Bone marrow aspirates were obtained from two patients diagnosed with AML who had been treated with FTI. These AML patients were administered 600 mg (B)-6-[amino(4-chlorophenyl)(1-methyl-1H-imidazol-5-yl)methyl]-4-(3-chlorophenyl)-1-methyl-2(1H)-quinolinone) twice daily over a 21 day period. Bone marrow aspirates were taken at baseline and once a week for the three weeks of treatment. One of these patients did not respond (RH) while the other responded (BS) to the FTI. Response was determined as a reduction of more than 50% of blast cells in bone marrow aspirates. The aspirates were diluted to 15 ml with PBS and Ficoll-density centrifuged. White blood cells were washed twice with PBS, resuspended in FBS with 10% DMSO and immediately frozen at −80° C. Cells were cryogenically preserved to maintain cell viability. Samples were thawed at 37° C. and 10× volume of RPMI with 20% FBS was added drop-wise over a period of 5 min. Cells were centrifuged at 1600 rpm for 10 min and resuspended in 10 ml PBS with 2 mM EDTA and 0.5% BSA. Samples were then passed through a 70 μM filter to remove any cell clumps. Cell viability was determined by Trypan Blue assay. If sample viability was less than 50% a Miltenyi Dead Cell Removal Kit was employed to enrich for the live cell fraction. 2×10⁵ viable cells were then double labeled with CD33-FITC and CD34-PE antibodies (Pharminigen) and FACS analysis was performed. Post (B)-6-[amino(4-chlorophenyl)(1-methyl-1H-imidazol-5-yl)methyl]-4-(3-chlorophenyl)-1-methyl-2(1H)-quinolinone)-treated bone marrow samples were enriched for leukemic cells by magnetic bead cell separation using either CD33 or CD34 antibodies (Miltenyi). The extracted cells had RNA extracted as described in Example 3.

### EXAMPLE 5

### Probe Preparation

RNA samples obtained in Examples 3 and 4 were prepared for hybridization to cDNA microarrays according to the following procedure. One to two rounds of linear amplification was performed on total RNA depending on the amount of starting material. Initially, 1-10 μg total RNA was reverse transcribed using the Superscript cDNA transcription kit (Gibco BRL). Ten μl total RNA was first mixed with 1 μl of 0.5 mg/ml T7-oligodT primer, incubated at 70° C. for 10 min, and then chilled on ice. Next, 8 μl of 5× first-strand reaction buffer, 0.1M DTT, 10 mM dNTPs, and 1 μl Rnase Block were added, and the solution incubated at 42° C. for 5 min. One μl Superscript II was then added and the reaction was incubated at 42° C. for 2 hr. The reaction was heat deactivted at 70° C. for 10 min and 1 μl was removed for PCR. Next, 92 μl Rnase-free water, 30 μl 5× second-strand reaction buffer, 3 μl 10 mM dNTP, 4 μl DNA polymerase 1, 1 μl E. coli Rnase H, 1 μl E. coli DNA ligase were added and the mixture incubated at 16° C. for 2 hr. cDNA was linear amplified using the Ampliscribe T7-transcription kit (Epicenter). If required, a second round of RNA amplification was performed by the random hexamer approach. Fluorescently labeled cDNA probes were synthesized by priming aRNA with random hexamers and including Cy3-dCTP in the nucleotide mix. Reactions were purified using a QIAquick PCR purification kit (Qiagen), the volumes of probe normalized using relative fluoresence (Cytofluor), and resuspended in 50 μl of Version 2 hybridization buffer (Amersham Pharmacia Biotech, Pistcataway, N.J.) with 50% formamide and human Cot1 DNA (Life Technologies).

### EXAMPLE 6

### Array Hybridization and Analysis

The arrays contained 7452 cDNAs from the IMAGE consortium (Integrated Molecular Analysis of Genome and their Expression: Research Genetics, Huntsville, Ala.) and Incyte libraries. Micro-arrays were generated as follows and probes hybridized as described in Example 5. cDNAs were printed on amino silane-coated slides (Corning) with a Generation III Micro-array Spotter (Molecular Dynamics). The cDNAs were PCR amplified, purified (Qiagen PCR purification kit), and mixed 1:1 with 10 M NaSCN printing buffer. Prior to hybridization micro-arrays were incubated in isopropanol at room temperature for 10 min. The probes were incubated at 95° C. for 2 min, at room temperature for 5 min, and then applied to three replicate slides. Cover slips were sealed onto the slides with DPX (Fluka) and incubated at 42° C. overnight. Slides were then washed at 55° C. for 5 min in 1×SSC/0.2% SDS and 0.1×SSC/0.2% SDS, dipped in 0.1×SSC and dried before being scanned by a GenIII Array Scanner (Molecular Dynamics). The fluorescence intensity for each spot was analyzed with AUTOGENE software (Biodiscovery, Los Angeles).

The intensity level of each micro-array was normalized so that the 75^{th} percentile of the expression levels was equal across micro-arrays. Clones displaying a coefficient of variance (CV) greater than 50% of the mean were excluded from the analysis. Since background intensity was a maximum of 32 units for all experiments a threshold of 32 was assigned to all clones exhibiting an expression level lower than this. A ratio matrix was then generated based on pair-wise analysis of treated and control samples and Hierarchical clustering was performed using an euclidean metric and average linkage (Omniviz Pro™).

Each sample was hybridized to three identical arrays and the mean signal intensity was compared by scatter-plot analysis. High correlation coefficients were also observed when control samples were compared to treated samples from the same day. This indicated there were no gross changes in gene expression due to treatment with (B)-6-[amino(4-chlorophenyl)(1-methyl-1H-imidazol-5-yl)methyl]-4-(3-chlorophenyl)-1-methyl-2(1H)-quinolinone). In addition, the variation between control samples from different days was examined. Cells were mock-treated and RNA was isolated after 1, 2, 3, 4, 5, and 6 days. Following labeling and hybridization the mean intensity of duplicate samples and the coefficient of variance (CV) of each clone (3 spots per clone) were calculated. Data points which displayed a CV of more than 50% were discarded from further analysis.

Genes analyzed according to this invention are identified by reference to Gene ID Numbers in the Genbank database. Where no such ID Numbers are available, nucleic acid sequences corresponding to the modulated genes are provided. These are typically related to full length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### EXAMPLE 7

### Differential Gene Expression in Treated Cell Line Samples

Hierarchical clustering was performed on the time-course data sets using the OmniViz Pro™ software (Battelle). Initially, fold-changes of 1.5, 1.7, and 2.0 were used as filters for the treated versus control intensity ratios for each day of the time-course. The gene expression profiles of genes modulated beyond these thresholds were analyzed to examine those genes that were commonly modulated between the three data sets and identify gene clusters that shared similar expression profiles. Results are shown in Tables 1-3 below.

### EXAMPLE 8

### Identification of Gene Networks

Genes that were regulated in two or more cell lines by at least 1.5-fold in drug treated cell lines (Table 1) were identified as described above. The list of these genes was employed to identify major gene pathways that were being modulated by the most preferred FTI, (B)-6-[amino(4-chlorophenyl)(1-methyl-1H-imidazol-5-yl)methyl]-4-(3-chlorophenyl)-1-methyl-2(1H)-quinolinone). If clones did not perfectly match a known gene annotations from the best BLAST result of the clone sequence were used. Since the level of regulation of these genes varied over the course of treatment of the cell lines, the gene expression profiles from the primary AML tissue that responded to this FTI were determined.

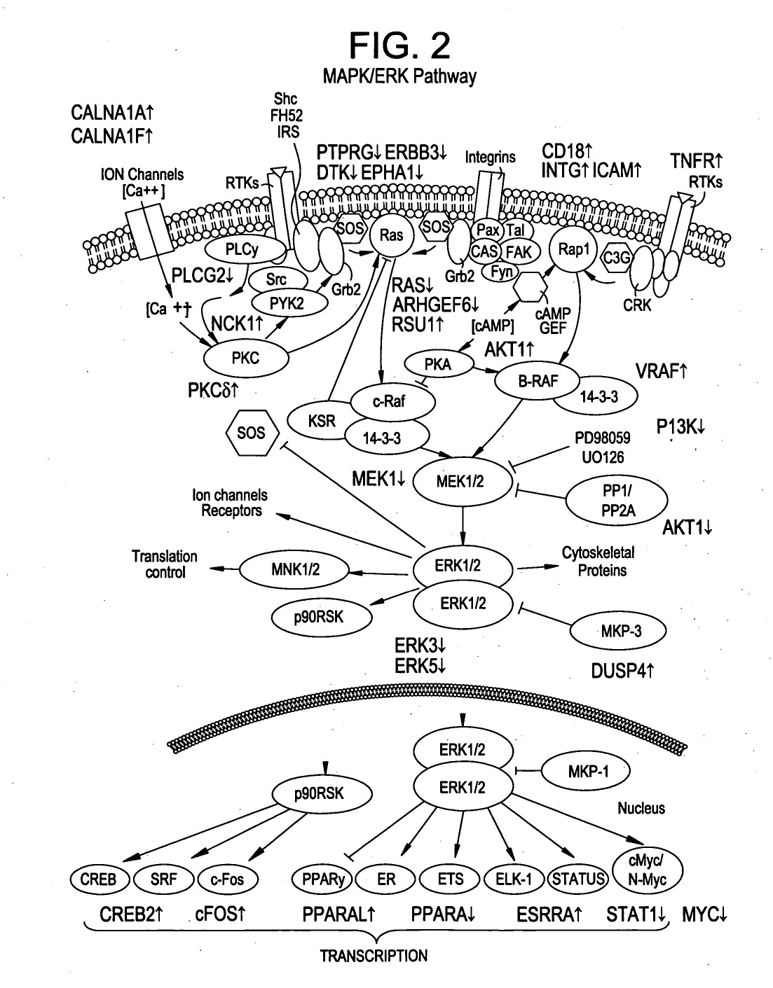

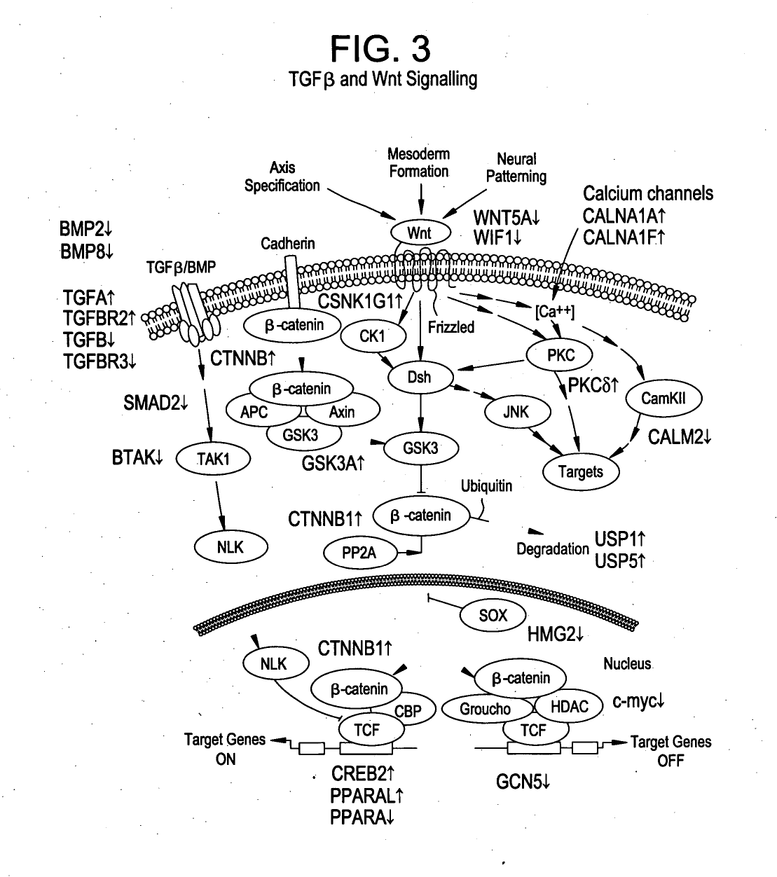

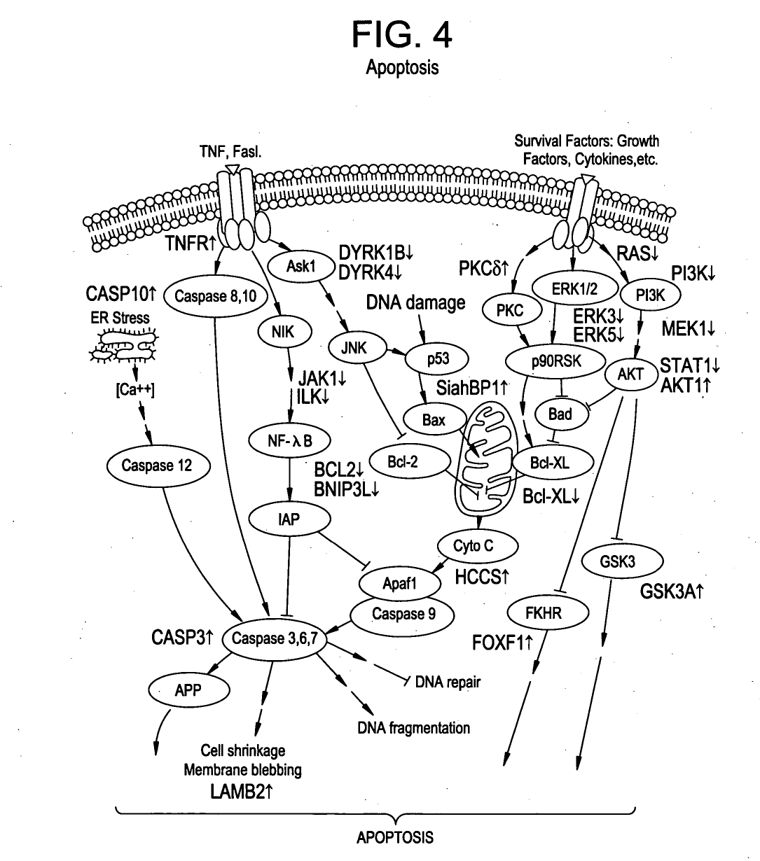

It was found that many genes in the MAPK/ERK (FIG. 2) signaling pathways were being down-regulated and that genes in the TGFβ (FIG. 3) and WNT (FIG. 3) signaling pathways were generally up-regulated, while apoptotic pathways were also activated (FIG. 4). This allowed the identification of other gene targets sensitive to treatment with known or novel drug compounds. For example, beneficial treatment can result from FTIs used in conjunction with tyrosine kinase, MEK kinase, PI3K and/or MAP kinase inhibitors to obtain a more potent effect. In addition, given the finding that apoptotic pathways are activated in FTI treated cells, drugs that modulate apoptosis could be expected to have beneficial effect when employed in conjunction with an FTI. Examples of these types of compounds include tyrosine kinase inhibitors (eg Gleevec, Novartis), MAP kinase inhibitors (eg U-0126, PD-098059, SB-203580), and inhibitors of anti-apoptotic genes such as Bcl-XL (eg antisense, ribozymes, DNAzymes).

TABLE 1
Genes (by Genbank Accession Number) modulated
at least 1.5 fold in 2 or more
of the cell lines over the 6 day time course.

| Gene ID | Accession No. | Seq ID |
|---|---|---|
| 3434105F7 | AB026898 | 846 |
| 3881595H1 | AC000134 | 691 |
| AI939481 | AC005155 | 870 |
| AA961061 | AC005670 | 879 |
| 3918104H1 | AC006023 | 706 |
| AI025519 | AC008427 | 529 |
| 5543360F8 | AC009220 | 738 |
| AA744682 | AC009289 | 498 |
| AA932129 | AC009756 | 523 |
| AI148008 | AC011473 | 882 |
| Y00052 | AC013722 | 54 |
| R52476 | AC021078 | 639 |
| AA864819 | AC022087 | 875 |
| AI815593 | AC022150 | 581 |
| AI141943 | AC026448 | 520 |
| AI300541 | AC073585 | 536 |
| 2515486H1 | AF161372 | 353 |
| 1731618H1 | AJ003147 | 822 |
| BE222911 | AJ400879 | 885 |
| R13802 | AK022901 | 807 |
| AI656222 | AL021155 | 898 |
| AI553823 | AL022313 | 775 |
| AA010251 | AL034397 | 46 |
| AA237071 | AL035420 | 467 |
| 2445101F6 | AL049824 | 328 |
| AI638342 | AL122004 | 575 |
| AA779424 | AL136980 | 492 |
| H81171 | AL137073 | 608 |
| R77754 | AL137790 | |
| AI209040 | AL139082 | 543 |
| 6421806H1 | AL139396 | 732 |
| H61066 | AL161787 | 605 |
| R59209 | AL355136 | 640 |
| AA486141 | AL355352 | 471 |
| AI339252 | AP001630 | 884 |
| AW023438 | BC009732 | 790 |
| 914979H1 | BC013834 | 419 |
| AA455969 | D00015 | 20 |
| T64335 | D00017 | 450 |
| X02308 | D00596 | 254 |
| X67098 | D00596 | 278 |
| X01023 | D10493 | 252 |
| D10493 | D10493 | 400 |
| Y00396 | D10493 | 395 |
| X69292 | D10667 | 279 |
| AA736561 | D11094 | 753 |
| S68252 | D11139 | 449 |
| M25315 | D12592 | 90 |
| AL186110 | D13118 | 532 |
| H84153 | D13639 | 153 |
| AA598561 | D14043 | 6 |
| D14695 | D14695 | 136 |
| 2206642T6 | D16889 | 351 |
| AI878943 | D17004 | 433 |
| U38846 | D17080 | 73 |
| J03801 | D21235 | 401 |
| AI762926 | D23660 | 565 |
| D25215 | D25215 | 138 |
| U41078 | D26512 | |
| AA485961 | D30648 | 470 |
| AA456408 | D38441 | 21 |
| AI311090 | D38616 | 555 |
| 1869911H1 | D42084 | 692 |
| D43950 | D43950 | 106 |
| AW006368 | D44467 | 585 |
| U29092 | D45050 | 235 |
| D45887 | D45887 | 437 |
| W68193 | D49489 | |
| 3633286H1 | D63874 | 415 |
| N76967 | D66904 | 201 |
| AA985407 | D82326 | 525 |
| AI962797 | D83260 | 593 |
| M59829 | D85730 | 82 |
| 3107995H1 | D86322 | 359 |
| AA279906 | D86550 | 481 |
| AI016874 | D86586 | 528 |
| 556963H1 | D86955 | 309 |
| AI810687 | D86997 | 788 |
| AF045581 | D87462 | 129 |
| U41070 | D89078 | 75 |
| D90209 | D90209 | 141 |
| AA812265 | D90767 | 501 |
| 2135769H1 | J02763 | 350 |
| 1876511H1 | J03072 | 643 |
| J04088 | J04088 | 156 |
| AI590075 | J04973 | 778 |
| H30357 | J05451 | 800 |
| K00558 | K03460 | 158 |
| L10413 | L00634 | 164 |
| L01087 | L01087 | 122 |
| AI027898 | L02426 | 516 |
| L06139 | L06139 | |
| U28936 | L07914 | 71 |
| M86400 | L07955 | 76 |
| AA428915 | L08634 | 424 |
| AA464627 | L09604 | 18 |
| M94859 | L10284 | 222 |
| R78541 | L10717 | |
| L15189 | L11066 | |
| L11284 | L11284 | |
| T87908 | L12387 | 452 |
| T80827 | L13802 | 451 |
| L08044 | L15203 | 163 |
| AI278029 | L16862 | 766 |
| M60278 | L17032 | 301 |
| M60278 | L17032 | 301 |
| M65128 | L18980 | 84 |
| W49672 | L20861 | 249 |
| L22005 | L22005 | 167 |
| AI380522 | L23822 | 552 |
| AA988469 | L25591 | 526 |
| L26336 | L26336 | 112 |
| AI074564 | L32866 | 539 |
| M63175 | L35233 | 194 |
| 2470939H1 | L35848 | 830 |
| AA190648 | L36055 | 423 |
| AI243166 | L38716 | 892 |
| L39833 | L39833 | 893 |
| M97347 | L41415 | 444 |
| 2005142H1 | L42324 | 348 |
| M11723 | L43615 | 101 |
| U04045 | L47574 | 231 |
| AA284067 | L76938 | 877 |
| M13142 | M13142 | 102 |
| W68291 | M15395 | 250 |
| 5560880H1 | M19483 | 861 |
| 3171275H1 | M20199 | 683 |
| M22489 | M22489 | 403 |
| AA070627 | M22810 | 421 |
| H39560 | M24736 | 146 |
| M25897 | M25897 | 91 |
| M27492 | M27492 | 181 |
| AA570304 | M29366 | |
| 205581R6 | M29696 | 349 |
| M84739 | M32294 | |
| M84739 | M32294 | |
| M35857 | M32315 | 189 |
| 3801801H1 | M33680 | 366 |
| M63904 | M63904 | 83 |
| AI091579 | M63971 | 518 |
| M69215 | M69215 | 85 |
| M74782 | M74782 | 216 |
| M80254 | M80254 | 86 |
| M80647 | M80647 | 218 |
| M84526 | M84526 | 88 |
| 6805226H1 | M84526 | 418 |
| S93414 | M86553 | 78 |
| M91556 | M91556 | 77 |
| M95678 | M95678 | 223 |
| 2017923F6 | M96326 | 825 |
| H73054 | NM_000551 | 151 |
| AA488324 | NM_001211 | 388 |
| N73242 | NM_001274 | 199 |
| AF013611 | NM_001335 | |
| AW163686 | NM_001524 | 589 |
| AI921879 | NM_002287 | 574 |
| AI652785 | NM_002333 | 554 |
| AI423526 | NM_003332 | 559 |
| 3028719F6 | NM_003600 | 680 |
| AB010882 | NM_003601 | 123 |
| AF030424 | NM_003642 | 126 |
| AW194791 | NM_003668 | 613 |
| AF075599 | NM_003969 | 132 |
| AF031141 | NM_004223 | 127 |
| 2676931T6 | NM_004412 | 312 |
| AF053304 | NM_004725 | 130 |
| AF119815 | NM_004885 | 399 |
| AI816398 | NM_004888 | 571 |
| 2185556H1 | NM_004917 | 326 |
| 3357511H1 | NM_004917 | |
| AA047585 | NM_005109 | 477 |
| AA448972 | NM_005592 | 503 |
| AW629084 | NM_005817 | 615 |
| AJ001015 | NM_005854 | 104 |
| AJ001016 | NM_005856 | 105 |
| U85055 | NM_006480 | 407 |
| 3618886F6 | NM_006536 | 319 |
| AA906714 | NM_006573 | 759 |
| AF060153 | NM_007037 | 398 |
| 1467864F6 | NM_012089 | |
| AI097079 | NM_012100 | 763 |
| AF204944 | NM_012105 | 527 |
| W44673 | NM_012428 | 670 |
| AI522316 | NM_013386 | 774 |
| AI700673 | NM_013439 | 577 |
| AA057781 | NM_014172 | 40 |
| AI299795 | NM_014251 | 547 |
| 1931159F6 | NM_014397 | 346 |
| AW630208 | NM_014413 | 795 |
| 2821685T6 | NM_014967 | 357 |
| 1539060H1 | NM_015343 | 334 |
| AI762738 | NM_015449 | 579 |
| AA401397 | NM_015596 | 384 |
| AW243944 | NM_015596 | 791 |
| AI436551 | NM_016141 | 553 |
| AI651159 | NM_016440 | 576 |
| AA527334 | NM_016625 | 508 |
| g922698 | NM_017555 | 380 |
| 1693028H1 | NM_017636 | 345 |
| 002783H1 | NM_017860 | 339 |
| AI368583 | NM_017874 | 429 |
| AW170305 | NM_017903 | 612 |
| H62827 | NM_018321 | 634 |
| M78706 | NM_019020 | 217 |
| BE048230 | NM_020216 | |
| AI214466 | NM_020334 | 535 |
| AA452802 | NM_021196 | 389 |
| AI808824 | NM_022082 | 787 |
| W77977 | NM_022336 | 815 |
| AA535015 | NM_022570 | 475 |
| AA861140 | NM_022829 | 521 |
| AW078834 | NM_023080 | 587 |
| 5122087H1 | NM_024056 | 369 |
| AF017182 | NM_024101 | 393 |
| AA449040 | NM_024116 | 483 |
| 2792728F6 | NM_024902 | 356 |
| BE218593 | NM_025230 | 630 |
| AA669885 | NM_030763 | 750 |
| AI339565 | NM_030908 | 767 |
| AF038564 | NM_031483 | 128 |
| AI126706 | NM_032038 | 519 |
| 1961084H1 | NM_032188 | 824 |
| 4609810F6 | NM_032554 | 713 |
| AA745592 | NM_032844 | 490 |
| AW612141 | NM_033050 | 793 |
| AI740538 | NM_033280 | 785 |
| U58522 | S51016 | 242 |
| M57703 | S63697 | 190 |
| AA873257 | S73591 | 758 |
| AA521213 | S77359 | 473 |
| N77754 | S79873 | 202 |
| AA984230 | S80071 | 524 |
| R79935 | S81439 | 213 |
| T71391 | T71391 | 665 |
| AA598776 | U05340 | 8 |
| U11053 | U11053 | 80 |
| T55353 | U12597 | 227 |
| AA456616 | U14970 | 22 |
| U18300 | U18300 | 234 |
| AF017306 | U20657 | 124 |
| AA459663 | U25182 | 19 |
| U27699 | U27699 | 880 |
| AI884916 | U29171 | 591 |
| U29171 | U29171 | 454 |
| 1671033F6 | U33429 | 337 |
| AA017042 | U40989 | 44 |
| AI126520 | U48405 | 542 |
| U48807 | U48807 | |
| U49395 | U49395 | 239 |
| AA186542 | U50078 | 464 |
| U51586 | U51586 | 240 |
| AW150605 | U54558 | 434 |
| AA455800 | U55206 | 25 |
| AF029777 | U57316 | 426 |
| I19355 | U58913 | 118 |
| 319095H1 | U58913 | 307 |
| AF027964 | U59911 | 125 |
| U60519 | U60519 | 62 |
| AI371158 | U65378 | 557 |
| U69883 | U69883 | 244 |
| H30148 | U73641 | 601 |
| R80718 | U75283 | 620 |
| U77180 | U77180 | 246 |
| U78180 | U78180 | 64 |
| U83115 | U83115 | 889 |
| AA938905 | U86218 | 514 |
| AI401546 | U88844 | 770 |
| 2526581H1 | U90904 | 650 |
| AA773114 | U95740 | 499 |
| AA176596 | U96781 | 478 |
| X13274 | V00543 | 258 |
| I16618 | V00595 | 155 |
| R91899 | X00226 | 660 |
| AW519155 | X00318 | 436 |
| X87344 | X00369 | 894 |
| X02910 | X01394 | |
| M15840 | X02532 | 172 |
| AA401046 | X02592 | 482 |
| X02812 | X02812 | |
| X87344 | X03066 | 894 |
| X03084 | X03084 | |
| M10901 | X03225 | 99 |
| X03225 | X03225 | 99 (?) |
| R81823 | X03742 | 447 |
| X04011 | X04011 | |
| K02400 | X04076 | 159 |
| X07036 | X04408 | 56 |
| X07036 | X04408 | 56 |
| Y00816 | X05309 | 293 |
| N20475 | X05344 | 225 |
| M11233 | X05344 | 100 |
| X02544 | X05784 | |
| X52192 | X06292 | |
| R33755 | X06547 | 207 |
| X06989 | X06989 | 256 |
| X07979 | X07979 | 257 |
| X14723 | X08004 | 259 |
| M20566 | X12830 | 178 |
| M20566 | X12830 | 178 |
| X13197 | X13197 | 408 |
| M21304 | X13709 | 179 |
| X00351 | X13839 | 251 |
| X60236 | X14008 | 410 |
| H57180 | X14034 | 148 |
| M33011 | X14758 | 441 |
| X14768 | X14768 | 58 |
| M31625 | X14768 | 96 |
| M31626 | X14768 | 97 |
| M30816 | X14768 | 95 |
| X52882 | X14983 | 59 |
| AA598758 | X15187 | 7 |
| X15606 | X15606 | 261 |
| K03515 | X16539 | 161 |
| AA868186 | X17093 | 425 |
| X51416 | X51416 | |
| M23699 | X51439 | 180 |
| AA455222 | X51675 | 24 |
| X51757 | X51757 | |
| X52195 | X52195 | 263 |
| U06434 | X53682 | 232 |
| J03198 | X54048 | 119 |
| M32304 | X54533 | 187 |
| AA487812 | X56134 | 9 |
| X56134 | X56134 | 265 |
| M16985 | X56257 | 174 |
| N31660 | X56257 | |
| H27379 | X57198 | 144 |
| X57522 | X57522 | 268 |
| X57830 | X57830 | 60 |
| M57765 | X58377 | 191 |
| X58528 | X58528 | 269 |
| M81182 | X58528 | 219 |
| S60489 | X60111 | 305 |
| AI739095 | X61157 | 563 |
| R76314 | X61587 | 212 |
| M83665 | X62534 | 404 |
| X65921 | X65921 | 277 |
| M37722 | X66945 | 98 |
| AA453816 | X69516 | 26 |
| AA187162 | X69654 | 422 |
| X69819 | X69711 | 280 |
| X70070 | X70070 | 61 |
| X70697 | X70697 | 281 |
| S40706 | X71427 | 303 |
| T53775 | X71874 | 226 |
| X71877 | X71877 | 47 |
| X73458 | X73458 | 283 |
| X74801 | X74801 | |
| AA454585 | X75755 | 2 |
| R43734 | X76939 | |
| AI189206 | X77303 | 533 |
| 2496221H1 | X77303 | 831 |
| H17504 | X80692 | |
| R26434 | X80910 | 205 |
| X83688 | X83688 | 285 |
| U24231 | X84709 | 302 |
| 3576337H1 | X85030 | 318 |
| X87212 | X87212 | 50 |
| T56477 | X87212 | 622 |
| AA464034 | X89401 | 16 |
| X89576 | X89576 | 51 |
| AA187458 | X92396 | 35 |
| M15887 | X94565 | 173 |
| X94991 | X94991 | 457 |
| X96427 | X96427 | 287 |
| X97058 | X97058 | |
| AA425120 | X98262 | 28 |
| 3283686H1 | XM_005825 | 684 |
| AW027188 | XM_005958 | 597 |
| 1525902F6 | XM_006646 | |
| R48796 | XM_008099 | 210 |
| AK000599 | XM_027140 | 584 |
| AA044653 | XM_031608 | 476 |
| AW665954 | XM_035574 | 617 |
| H86407 | XM_037453 | 802 |
| R00285 | XM_038150 | 609 |
| X57447 | XM_039395 | 267 |
| 778372H1 | XM_040459 | 375 |
| R82530 | XM_041024 | 448 |
| AI580830 | XM_042041 | 562 |
| 3097063H1 | XM_044784 | |
| 3038910H1 | XM_046691 | 358 |
| H53340 | XM_048213 | 147 |
| 1654210F6 | XM_048530 | 336 |
| L42856 | XM_054964 | 168 |
| 2707270F6 | XM_056259 | 355 |
| M23468 | Y00062 | |
| Y00649 | Y00649 | 291 |
| Y00757 | Y00757 | 292 |
| M17017 | Y00787 | 175 |
| M28130 | Y00787 | 92 |
| L02932 | Y07619 | 108 |
| Y10256 | Y10256 | 294 |
| AA454813 | Y12395 | 1 |
| AA149850 | Y12670 | 466 |
| 704183H1 | Y13710 | |
| 059476H1 | Y13829 | 340 |
| Y13834 | Y13834 | 458 |
| L11016 | Y14768 | 165 |
| 3141315H1 | Y17803 | 361 |
| AI341167 | Y18391 | 768 |
| AI707852 | Z12962 | 432 |
| U51278 | Z23115 | 394 |
| AI686653 | Z26876 | 430 |
| AA043102 | Z35102 | 459 |
| AA136533 | Z35481 | 381 |
| U49083 | Z49148 | 455 |
| R70234 | Z56852 | 446 |
| 257274R6 | Z58168 | 354 |
| U62027 | Z73157 | 63 |
| 391237F1 | Z73157 | 367 |
| 510997F1 | Z73157 | 368 |
| AI808621 | Z82214 | 569 |
| AA460801 | Z98749 | |
| 2673259F6 | Z98752 | 699 |
| R22977 | Z98946 | 204 |
| L03380 | Z99995 | 402 |
| AA425422 | | 29 |
| AA460392 | | 504 |
| AA508510 | | 472 |
| AA552028 | | 509 |
| AA576785 | | 510 |
| AA663307 | | 748 |
| AI015248 | | 872 |
| AI024468 | | 896 |
| AI086865 | | 540 |
| AI190605 | | 897 |
| AI203269 | | 871 |
| AI264420 | | 545 |
| AI333013 | | 556 |
| AI435052 | | 771 |
| AI671268 | | 781 |
| AI796718 | | 568 |
| AI990816 | | 594 |
| AW027164 | | 596 |
| AW167520 | | 891 |
| H24679 | | 633 |
| H66015 | | 636 |
| H91370 | | 637 |
| W07570 | | 667 |
| 1274737F6 | | 672 |
| 195337H1 | | 347 |
| 2398102H1 | | 647 |
| 2531082H1 | | 698 |
| 264639H1 | | 329 |
| 2794246F6 | | 654 |
| 3290073H1 | | 685 |
| 335737H1 | | 362 |
| 4539942F8 | | 711 |
| 6300669H1 | | 417 |
| 938765H1 | | |

TABLE 2
Genes (by GenBank Accession Number)
modulated at least 1.7 fold in primary AML Sample.

| Gene ID | Accession No. | Seq ID |
|---|---|---|
| T94331 | AB026898 | 810 |
| 3881595H1 | AC000134 | 691 |
| 1329021F6 | AC002073 | 816 |
| 2858615H1 | AC002325 | 836 |
| AI791539 | AC002428 | 566 |
| AI821217 | AC004258 | 582 |
| AA774798 | AC004671 | 868 |
| H29666 | AC004845 | 600 |
| T95173 | AC005071 | 811 |
| AA814523 | AC005160 | 887 |
| 5905620T9 | AC005212 | 731 |
| 5986963H1 | AC005280 | 865 |
| 5825251H1 | AC005306 | 864 |
| N36113 | AC005670 | 886 |
| 1700438H1 | AC005682 | 412 |
| R48756 | AC005757 | 638 |
| 5538589F6 | AC005839 | 859 |
| 3918104H1 | AC006023 | 706 |
| AA443719 | AC007240 | 468 |
| AI867297 | AC007883 | 590 |
| R63067 | AC008073 | 659 |
| AW022174 | AC008382 | 595 |
| 5537789F6 | AC008525 | 721 |
| H00249 | AC008733 | 599 |
| 1436240H1 | AC008860 | 674 |
| 2668191F6 | AC008949 | 833 |
| 5543360F8 | AC009220 | 738 |
| AA737674 | AC009892 | 881 |
| 5104579H1 | AC009892 | 718 |
| 3335217F6 | AC010311 | 686 |
| BE326380 | AC010521 | 631 |
| 3746214H1 | AC011088 | 689 |
| 1671315F6 | AC011500 | 820 |
| H60969 | AC012351 | 604 |
| AA926944 | AC012377 | 512 |
| 3100089H1 | AC012454 | 682 |
| Y00052 | AC013722 | 54 |
| R52476 | AC021078 | 639 |
| N45149 | AC021106 | 803 |
| AI742120 | AC022137 | 564 |
| 4177228F6 | AC022224 | 855 |
| 2676312H1 | AC022415 | 652 |
| H73476 | AC022740 | 607 |
| AA652121 | AC046170 | 487 |
| AI308320 | AC046170 | 890 |
| 1956982H1 | AC046170 | 645 |
| 1428534F6 | AC051619 | 818 |
| 2914934H1 | AC055707 | 701 |
| AA621370 | AC064807 | 511 |
| 5514511R6 | AC073333 | |
| AI698737 | AC074331 | 783 |
| 3406131H1 | AC079118 | 724 |
| N20072 | AC096579 | 224 |
| 5911413H1 | AC096667 | |
| AI458182 | AF042782 | 773 |
| 2291436H1 | AF074333 | 646 |
| W32067 | AF136745 | 669 |
| 6755801J1 | AF157623 | 746 |
| 2397317F6 | AF235100 | 827 |
| R53190 | AF384819 | 619 |
| 1731618H1 | AJ003147 | 822 |
| 2959801H1 | AJ003147 | 703 |
| 3123232H1 | AJ003147 | 840 |
| 2760110H1 | AJ006345 | 314 |
| X64073 | AJ239325 | 274 |
| 3986782F7 | AJ249275 | 850 |
| AI366098 | AJ276674 | 769 |
| AI695385 | AJ289236 | 899 |
| BE222911 | AJ400879 | 885 |
| AI400473 | AK017738 | 558 |
| AI299633 | AK021499 | 546 |
| R13802 | AK022901 | 807 |
| 1489075H1 | AK025775 | 343 |
| AI656222 | AL021155 | 898 |
| W96144 | AL021155 | 626 |
| 2459540H1 | AL031282 | 829 |
| 3461693F6 | AL031588 | 687 |
| 4333034H1 | AL031726 | 709 |
| 3332309H1 | AL031728 | 705 |
| R61661 | AL032821 | 658 |
| U71321 | AL033519 | 406 |
| AA935151 | AL034374 | 513 |
| AA010251 | AL034397 | 46 |
| U43431 | AL035367 | 238 |
| AA237071 | AL035420 | 467 |
| AA609779 | AL049610 | 114 |
| AA167461 | AL049612 | 463 |
| 4228729H2 | AL049742 | 857 |
| 6712339H1 | AL049766 | 867 |
| AI051176 | AL049872 | 531 |
| 1747028H1 | AL078600 | 642 |
| 5164454H1 | AL109840 | 370 |
| 7007735H1 | AL117382 | 742 |
| AA526337 | AL121601 | 495 |
| AI638342 | AL122004 | 575 |
| 4835576H1 | AL122035 | 715 |
| W01596 | AL133243 | 812 |
| U64205 | AL133367 | |
| 4820983H1 | AL135786 | 714 |
| 5594552H1 | AL136381 | 723 |
| H12102 | AL136979 | 798 |
| H81171 | AL137073 | 608 |
| AA151374 | AL137790 | 37 |
| AA578089 | AL138787 | 496 |
| AI209040 | AL139082 | 543 |
| 6421806H1 | AL139396 | 732 |
| H60498 | AL157776 | 603 |
| 3721604H1 | AL160271 | |
| H61066 | AL161787 | 605 |
| 2798009H1 | AL162252 | 834 |
| 2225447F6 | AL162430 | 695 |
| AI885557 | AL162729 | 573 |
| 2918417F6 | AL163279 | 657 |
| AA489975 | AL355151 | 505 |
| U77456 | AL355794 | 247 |
| 5375277T9 | AL356266 | 735 |
| AI051860 | AL356489 | 517 |
| 4019605F6 | AL356489 | 851 |
| U29607 | AL356801 | 236 |
| AA861429 | AL359512 | 757 |
| AA767859 | AL359915 | 756 |
| 1362587H1 | AL391122 | 627 |
| R09122 | AL391194 | 806 |
| R93094 | AP000173 | 662 |
| AA954331 | AP000432 | 760 |
| R10535 | AP000555 | 611 |
| 5327443H1 | AP000936 | 720 |
| 1569726H1 | AP001347 | 676 |
| R92422 | AP001672 | 661 |
| 3422674H1 | AP002800 | 364 |
| AI310451 | AP002812 | 550 |
| 3568042H1 | AP003900 | 725 |
| AA455969 | D00015 | 20 |
| AF030575 | D00015 | 427 |
| T64335 | D00017 | 450 |
| D12614 | D00102 | 135 |
| X67098 | D00596 | 278 |
| R27585 | D00759 | 206 |
| AA465593 | D00762 | 15 |
| M80436 | D10202 | 87 |
| M80436 | D10202 | 87 |
| M80436 | D10202 | 87 |
| M80436 | D10202 | 87 |
| AA464600 | D10493 | 17 |
| AI147046 | D10653 | 764 |
| S68252 | D11139 | 449 |
| M25315 | D12592 | 90 |
| AI186110 | D13118 | 532 |
| S57708 | D13515 | 304 |
| D13626 | D13626 | |
| AA682625 | D13641 | 497 |
| AA598561 | D14043 | 6 |
| D14695 | D14695 | 136 |
| D14825 | D14825 | 137 |
| 855326R1 | D16234 | |
| L20046 | D16305 | 166 |
| V00496 | D17206 | 456 |
| AA629808 | D17554 | 382 |
| M57285 | D21214 | 81 |
| J03801 | D21235 | 401 |
| AI700360 | D21878 | 431 |
| D25216 | D25216 | 139 |
| U41078 | D26512 | |
| AF245447 | D28468 | 515 |
| AF245447 | D28468 | 515 |
| AA070997 | D29012 | 43 |
| 2134847H1 | D30756 | 324 |
| AI147295 | D30756 | 428 |
| AA455067 | D31839 | 23 |
| AI311090 | D38616 | 555 |
| AW629690 | D42084 | 794 |
| 1869911H1 | D42084 | 692 |
| 5122374H1 | D43701 | 719 |
| D43950 | D43950 | 106 |
| D45887 | D45887 | 437 |
| W68193 | D49489 | |
| X72498 | D50326 | 282 |
| L11667 | D63861 | 110 |
| D63874 | D63874 | 140 |
| 3633286H1 | D63874 | 415 |
| X61598 | D83174 | |
| AA279906 | D86550 | 481 |
| AA729988 | D86550 | 752 |
| D86956 | D86956 | |
| L36719 | D87116 | 440 |
| D89078 | D89078 | 107 |
| U41070 | D89078 | 75 |
| AI821897 | D89675 | 789 |
| D90209 | D90209 | 141 |
| 2135769H1 | J02763 | 350 |
| J03040 | J03040 | 438 |
| 1876511H1 | J03072 | 643 |
| J03258 | J03258 | 120 |
| J03571 | J03571 | 296 |
| J04111 | J04111 | 157 |
| AI125073 | J04132 | 541 |
| 1634342H1 | J04794 | 677 |
| H30357 | J05451 | 800 |
| K02054 | K02054 | 297 |
| X02415 | K02569 | 255 |
| K03000 | K03000 | 160 |
| H58873 | K03195 | 149 |
| AI791949 | K03474 | 567 |
| L10413 | L00634 | 164 |
| H22919 | L03558 | 143 |
| L04288 | L04288 | |
| AA405769 | L05144 | 30 |
| H62473 | L07594 | 150 |
| L08177 | L08177 | 109 |
| L08177 | L08177 | 109 |
| AA234897 | L08895 | 36 |
| AA464627 | L09604 | 18 |
| M94859 | L10284 | 222 |
| R78541 | L10717 | |
| L15189 | L11066 | |
| M15400 | L11910 | 171 |
| L12168 | L12168 | 439 |
| L12350 | L12350 | 298 |
| L12350 | L12350 | 298 |
| T87908 | L12387 | 452 |
| L09600 | L13974 | |
| M14221 | L16510 | 103 |
| M60278 | L17032 | 301 |
| M60278 | L17032 | 301 |
| M60278 | L17032 | 301 |
| M60278 | L17032 | 301 |
| 2745317H1 | L17411 | 653 |
| M65128 | L18980 | 84 |
| W49672 | L20861 | 249 |
| AI380522 | L23822 | 552 |
| AA988469 | L25591 | 526 |
| NM_001168 | L26245 | 445 |
| R20939 | L31848 | 618 |
| 2470939H1 | L35848 | 830 |
| AA442810 | L36034 | 502 |
| L36148 | L36148 | 113 |
| M11723 | L43615 | 101 |
| M14745 | M14745 | 170 |
| W68291 | M15395 | 250 |
| M16038 | M16038 | |
| 339598H1 | M16038 | |
| M17783 | M17783 | 176 |
| 3171275H1 | M20199 | 683 |
| 5189380H1 | M21121 | 734 |
| 4130807F7 | M22440 | 854 |
| M22612 | M22612 | 299 |
| AA070627 | M22810 | 421 |
| 1445982H1 | M23254 | 342 |
| M28638 | M24906 | 93 |
| R45525 | M28215 | 209 |
| AI051962 | M28983 | 762 |
| 736837R6 | M29696 | 373 |
| M29870 | M29870 | 182 |
| AI264247 | M30309 | 876 |
| 1512407F6 | M30310 | 629 |
| M30471 | M30471 | 184 |
| M30704 | M30703 | 185 |
| AW467649 | M31158 | 435 |
| M84739 | M32294 | |
| M84739 | M32294 | |
| U52165 | M32315 | 241 |
| M35857 | M32315 | 189 |
| 5077322H1 | M32315 | 416 |
| N72918 | M34175 | 198 |
| M63193 | M58602 | 443 |
| M59465 | M59465 | 193 |
| 2294719H1 | M60858 | 352 |
| 2992331H1 | M63005 | 839 |
| AA069596 | M63582 | 42 |
| M63904 | M63904 | 83 |
| AI091579 | M63971 | 518 |
| M74782 | M74782 | 216 |
| AA410680 | M77016 | 31 |
| M80647 | M80647 | 218 |
| M84526 | M84526 | 88 |
| S93414 | M86553 | 78 |
| AI310138 | M91463 | 549 |
| M95678 | M95678 | 223 |
| 2017923F6 | M96326 | 825 |
| R60624 | NM_000702 | |
| AA488324 | NM_001211 | 388 |
| AA488341 | NM_001336 | 386 |
| AF006823 | NM_002246 | |
| 1322305T6 | NM_002250 | 332 |
| AI921879 | NM_002287 | 574 |
| AW129770 | NM_002349 | 588 |
| AJ004977 | NM_002873 | 134 |
| AI423526 | NM_003332 | 559 |
| 4516963H1 | NM_003576 | 710 |
| 3028719F6 | NM_003600 | 680 |
| AB010882 | NM_003601 | 123 |
| AF030424 | NM_003642 | 126 |
| AF029899 | NM_003814 | 397 |
| AF055993 | NM_003864 | 131 |
| AI220935 | NM_004142 | 765 |
| AW665782 | NM_004142 | 616 |
| AI191941 | NM_004226 | 534 |
| 1392516T6 | NM_004621 | 333 |
| AA449579 | NM_004769 | 116 |
| 1810447H1 | NM_004917 | 321 |
| AA047585 | NM_005109 | 477 |
| 4181072F6 | NM_005468 | 856 |
| AA448972 | NM_005592 | 503 |
| AA742351 | NM_005739 | 754 |
| 3406436F6 | NM_005845 | |
| AJ001015 | NM_005854 | 104 |
| 3118530H1 | NM_005880 | 360 |
| AA906714 | NM_006573 | 759 |
| AI016020 | NM_006672 | 761 |
| AW770551 | NM_006770 | 796 |
| AW009940 | NM_006871 | 586 |
| 864164H1 | NM_007194 | 311 |
| 1467864F6 | NM_012089 | |
| AF204944 | NM_012105 | 527 |
| W23427 | NM_012115 | 624 |
| 3363678H2 | NM_012226 | 363 |
| AI652076 | NM_012243 | 780 |
| 346874T6 | NM_013308 | 365 |
| AI522316 | NM_013386 | 774 |
| AI338030 | NM_013439 | 537 |
| AI700673 | NM_013439 | 577 |
| 4540025H1 | NM_014322 | 320 |
| W00842 | NM_014331 | 623 |
| AW511388 | NM_014358 | 614 |
| AW630208 | NM_014413 | 795 |
| H63640 | NM_014834 | 635 |
| AI743175 | NM_014959 | 786 |
| 2821685T6 | NM_014967 | 357 |
| W38474 | NM_015542 | 814 |
| AW243944 | NM_015596 | 791 |
| W07181 | NM_015701 | 813 |
| 2997457H1 | NM_015938 | 316 |
| AA631149 | NM_016205 | 485 |
| AA527334 | NM_016625 | 508 |
| 5543749F6 | NM_017414 | 739 |
| AW170305 | NM_017903 | 612 |
| AA160974 | NM_018155 | 462 |
| AA625433 | NM_018404 | 484 |
| AA074666 | NM_018834 | 38 |
| 767295H1 | NM_018983 | 374 |
| M78706 | NM_019020 | 217 |
| AF245447 | NM_020126 | 515 |
| AF245447 | NM_020126 | 515 |
| 4294821H1 | NM_020344 | 858 |
| 2490994H1 | NM_021624 | 697 |
| 3556218H1 | NM_021634 | 317 |
| 2435705R6 | NM_022048 | 648 |
| 3092423H1 | NM_022054 | 413 |
| W77977 | NM_022336 | 815 |
| AA429219 | NM_023930 | 27 |
| 1001514R6 | NM_024022 | 330 |
| AI031531 | NM_024083 | 530 |
| AA449040 | NM_024116 | 483 |
| 2803571H1 | NM_024586 | 315 |
| 1390130H1 | NM_024671 | 817 |
| 3241088H1 | NM_024850 | 842 |
| H96170 | NM_030779 | 117 |
| 1540906H1 | NM_030779 | 335 |
| AI824146 | NM_030811 | 583 |
| W90438 | NM_032127 | 625 |
| AA430653 | NM_032177 | 390 |
| 3495438F6 | NM_032294 | 847 |
| AW612141 | NM_033050 | 793 |
| AA417237 | NM_033225 | |
| AI740538 | NM_033280 | 785 |
| M57703 | S63697 | 190 |
| 780099H1 | S63912 | 376 |
| AA714835 | S67156 | 878 |
| AA777347 | S76736 | 491 |
| AA521213 | S77359 | 473 |
| U39231 | S79852 | 74 |
| N77754 | S79873 | 202 |
| AA984230 | S80071 | 524 |
| U00672 | U00672 | 229 |
| U02478 | U02478 | 230 |
| AA019459 | U02680 | 45 |
| 3401107H1 | U03019 | 845 |
| AI580044 | U04816 | 777 |
| 3041874H1 | U07563 | 704 |
| 2457652H1 | U12465 | 649 |
| U39318 | U13175 | 237 |
| U13666 | U13666 | 67 |
| U13695 | U13695 | 453 |
| AA056652 | U14176 | 460 |
| AA456616 | U14970 | 22 |
| U18242 | U18242 | 68 |
| U18300 | U18300 | 234 |
| AA465444 | U18422 | 14 |
| U20537 | U20536 | 69 |
| U25128 | U25128 | 70 |
| U35237 | U26174 | 72 |
| AI884916 | U29171 | 591 |
| AA481076 | U31278 | 13 |
| NM_002411 | U33147 | 405 |
| 1671033F6 | U33429 | 337 |
| AA664389 | U35048 | 4 |
| 6313632H1 | U43030 | 744 |
| R09288 | U43522 | 610 |
| AA488645 | U47007 | 10 |
| U47077 | U47077 | 873 |
| 5801413H1 | U48449 | 730 |
| 2405358R6 | U48729 | 828 |
| AA186542 | U50078 | 464 |
| U51586 | U51586 | 240 |
| 1355140F1 | U51586 | |
| AA455800 | U55206 | 25 |
| U56390 | U56390 | |
| U83410 | U58088 | 65 |
| AA121261 | U58675 | 461 |
| AF027964 | U59911 | 125 |
| U60519 | U60519 | 62 |
| 2836805T6 | U62293 | 656 |
| U62433 | U62433 | 243 |
| 3188135H1 | U66673 | 306 |
| 3188135H1 | U66673 | |
| 3188135H1 | U66673 | |
| 3188135H1 | U66673 | |
| 1360938T6 | U66679 | 341 |
| 809631T6 | U66684 | 377 |
| AA454652 | U67058 | 3 |
| AI214335 | U68755 | 544 |
| U69883 | U69883 | 244 |
| R98589 | U81375 | 663 |
| 5695322H1 | U82671 | 741 |
| AA745989 | U82979 | 755 |
| AA188256 | U83661 | 479 |
| 2526581H1 | U90904 | 650 |
| AA434064 | U95000 | 385 |
| AA773114 | U95740 | 499 |
| AA514978 | U96776 | 506 |
| Y07503 | V00510 | 411 |
| X96754 | V00557 | 288 |
| N67917 | V01512 | 197 |
| V01514 | V01514 | 66 |
| X87344 | X00369 | 894 |
| N53169 | X00567 | 196 |
| X02910 | X01394 | |
| X01451 | X01451 | 253 |
| X01451 | X01451 | 253 |
| X01451 | X01451 | 253 |
| X01451 | X01451 | 253 |
| AA401046 | X02592 | 482 |
| 5537736F6 | X02592 | 736 |
| X87344 | X03066 | 894 |
| M10901 | X03225 | 99 |
| M54894 | X04403 | 300 |
| M54894 | X04403 | 300 |
| M54894 | X04403 | 300 |
| M54894 | X04403 | 300 |
| X07036 | X04408 | 56 |
| X07036 | X04408 | 56 |
| N75719 | X04744 | 200 |
| M19507 | X04876 | 177 |
| Y00816 | X05309 | 293 |
| M11233 | X05344 | 100 |
| AA479102 | X05972 | 12 |
| N24824 | X06182 | |
| R33755 | X06547 | 207 |
| N41062 | X06820 | 195 |
| M86511 | X06882 | 221 |
| X07549 | X07549 | 57 |
| 1686702H1 | X07730 | 821 |
| X07979 | X07979 | 257 |
| X14723 | X08004 | 259 |
| J03561 | X12510 | 121 |
| J03561 | X12510 | 121 |
| J03561 | X12510 | 121 |
| J03561 | X12510 | 121 |
| M20566 | X12830 | 178 |
| M20566 | X12830 | 178 |
| M20566 | X12830 | 178 |
| M20566 | X12830 | 178 |
| U76549 | X12882 | 245 |
| M21304 | X13709 | 179 |
| X00351 | X13839 | 251 |
| X14830 | X14830 | 260 |
| X52882 | X14983 | 59 |
| AA598758 | X15187 | 7 |
| H27564 | X15729 | 145 |
| W15277 | X15940 | 248 |
| AA393214 | X15949 | 33 |
| M23502 | X16166 | 89 |
| K03515 | X16539 | 161 |
| M28880 | X16609 | 94 |
| 2403512H1 | X16674 | 327 |
| AA868186 | X17093 | 425 |
| J03236 | X51345 | 392 |
| X51416 | X51416 | |
| AA411440 | X51521 | 32 |
| AA058828 | X51602 | 41 |
| AA455222 | X51675 | 24 |
| X51804 | X51804 | 262 |
| T72877 | X52015 | 228 |
| X52195 | X52195 | 263 |
| X52947 | X52947 | 409 |
| U06434 | X53682 | 232 |
| 3081284F6 | X53702 | 681 |
| M36821 | X53799 | |
| AA490256 | X54048 | 11 |
| J03198 | X54048 | 119 |
| M60761 | X54228 | 442 |
| M11025 | X55283 | 169 |
| M33294 | X55313 | 188 |
| M33294 | X55313 | 188 |
| M31627 | X55543 | 186 |
| X55544 | X55544 | 264 |
| AA487812 | X56134 | 9 |
| X56134 | X56134 | 265 |
| X56777 | X56777 | 266 |
| H27379 | X57198 | 144 |
| M83652 | X57748 | 220 |
| X58528 | X58528 | 269 |
| M81182 | X58528 | 219 |
| S60489 | X60111 | 305 |
| X60592 | X60592 | 270 |
| R76314 | X61587 | 212 |
| M83665 | X62534 | 404 |
| R11490 | X62947 | 203 |
| AI436567 | X63422 | 560 |
| X63465 | X63465 | 271 |
| AA083577 | X63527 | 39 |
| X63547 | X63546 | 272 |
| 2159360H1 | X63692 | 325 |
| X64074 | X63926 | 275 |
| X63926 | X63926 | 275/276 (?) |
| X64083 | X63926 | 276 |
| 2535659H1 | X69168 | 832 |
| AA187162 | X69654 | 422 |
| X69819 | X69711 | 280 |
| AI310990 | X71491 | 551 |
| T53775 | X71874 | 226 |
| 3285272H1 | X73568 | 414 |
| U11087 | X75299 | 233 |
| X75299 | X75299 | 48 |
| AA454585 | X75755 | 2 |
| X75821 | X75821 | 49 |
| X75918 | X75918 | |
| X76029 | X76029 | 284 |
| R43734 | X76939 | 208 |
| AI189206 | X77303 | 533 |
| H17504 | X80692 | 142 |
| R26434 | X80910 | 205 |
| AI521155 | X81892 | 561 |
| AA088861 | X83228 | 383 |
| U10440 | X84849 | 79 |
| 407169H1 | X84909 | 852 |
| 3576337H1 | X85030 | 318 |
| T55802 | X85117 | 664 |
| 4407508H1 | X85337 | 728 |
| AA025432 | X85373 | 420 |
| T56477 | X87212 | 622 |
| AA464034 | X89401 | 16 |
| X89576 | X89576 | 51 |
| X89576 | X89576 | 51 |
| R83270 | X89750 | 214 |
| 917064H1 | X91249 | 378 |
| X91809 | X91809 | 286 |
| X92106 | X92106 | 52 |
| AA187458 | X92396 | 35 |
| AJ000519 | X92962 | 133 |
| X94991 | X94991 | 457 |
| X96427 | X96427 | 287 |
| R85213 | X98022 | 215 |
| X98296 | X98296 | 289 |
| X99585 | X99585 | 53 |
| R48796 | XM_008099 | 210 |
| R50354 | XM_009915 | 211 |
| W15172 | XM_016514 | 668 |
| AK000599 | XM_027140 | 584 |
| 7157414H1 | XM_031246 | 747 |
| AA044653 | XM_031608 | 476 |
| L16953 | XM_032556 | 111 |
| 1266202T6 | XM_033674 | 331 |
| AA805691 | XM_033788 | 500 |
| AA861582 | XM_036492 | 522 |
| H86407 | XM_037453 | 802 |
| 778372H1 | XM_040459 | 375 |
| AA016239 | XM_041087 | 895 |
| AI580830 | XM_042041 | 562 |
| AI732875 | XM_042637 | 578 |
| AA463411 | XM_045320 | 387 |
| AA648280 | XM_046411 | 115 |
| 3038910H1 | XM_046691 | 358 |
| H63831 | XM_047328 | 606 |
| 1654210F6 | XM_048530 | 336 |
| AA460131 | XM_049228 | 469 |
| 5539620F6 | XM_049755 | 722 |
| AA682896 | XM_050250 | 488 |
| L42856 | XM_054964 | 168 |
| 1483347H1 | XM_056259 | 628 |
| AI307255 | XM_058135 | 548 |
| H74265 | Y00062 | 152 |
| Y00064 | Y00064 | 290 |
| M17017 | Y00787 | 175 |
| M28130 | Y00787 | 92 |
| L02932 | Y07619 | 108 |
| AA504415 | Y09781 | 494 |
| AI809036 | Y12336 | 570 |
| AA516206 | Y12851 | 507 |
| 000527H1 | Y13829 | 338 |
| 059476H1 | Y13829 | 340 |
| Y13834 | Y13834 | 458 |
| L11016 | Y14768 | 165 |
| 3141315H1 | Y17803 | 361 |
| 551234R6 | Y17803 | 308 |
| AA426103 | Y18000 | 396 |
| H97778 | Z13009 | 154 |
| AA402431 | Z15005 | 34 |
| L07555 | Z22576 | 162 |
| U51278 | Z23115 | 394 |
| M58525 | Z26491 | 192 |
| AW772610 | Z26652 | 797 |
| Z29090 | Z29090 | 295 |
| H19371 | Z32684 | 632 |
| AA136533 | Z35481 | 381 |
| Z48810 | Z48810 | 55 |
| U49083 | Z49148 | 455 |
| R70234 | Z56852 | 446 |
| 4902714H1 | Z69918 | 716 |
| 150224T6 | Z80147 | |
| M29871 | Z82188 | 183 |
| AI808621 | Z82214 | 569 |
| AA699919 | Z83821 | 874 |
| 5538394H1 | Z83843 | 737 |
| 5020377F9 | Z97832 | 717 |
| AA460801 | Z98749 | |
| AI625585 | Z98750 | 779 |
| 2673259F6 | Z98752 | 699 |
| R22977 | Z98946 | 204 |
| AA007595 | | 869 |
| AA188574 | | 480 |
| AA280754 | | 465 |
| AA283874 | | 391 |
| AA460392 | | 504 |
| AA508510 | | 472 |
| AA515469 | | 5 |
| AA526772 | | 474 |
| AA576785 | | 510 |
| AA634241 | | 486 |
| AA663307 | | 748 |
| AA663482 | | 749 |
| AA713864 | | 751 |
| AA714520 | | 489 |
| AA828809 | | 493 |
| AA868502 | | 883 |
| AI061445 | | 538 |
| AI086865 | | 540 |
| AI264420 | | 545 |
| AI378131 | | 888 |
| AI440504 | | 772 |
| AI567491 | | 776 |
| AI693066 | | 782 |
| AI709066 | | 784 |
| AI766478 | | 580 |
| AI821337 | | 572 |
| AI949694 | | 592 |
| AW439329 | | 792 |
| AW630054 | | 598 |
| H24679 | | 633 |
| H29257 | | 799 |
| H51856 | | 602 |
| H66015 | | 636 |
| H72339 | | 801 |
| N57580 | | 805 |
| N54592 | | 804 |
| W07570 | | 667 |
| T75463 | | 900 |
| R88730 | | 808 |
| R91509 | | 809 |
| T56441 | | 621 |
| T77711 | | 666 |
| W92423 | | 671 |
| 1274737F6 | | 672 |
| 1338107F6 | | 673 |
| 1508571F6 | | 675 |
| 1548205H1 | | 344 |
| 1594182F6 | | 819 |
| 1594701F6 | | 641 |
| 1879290H1 | | 823 |
| 1902928H1 | | 644 |
| 194370H1 | | 322 |
| 195337H1 | | 347 |
| 198381H1 | | 323 |
| 2021568H1 | | 693 |
| 205203T6 | | 694 |
| 2194064H1 | | 826 |
| 224922R6 | | 696 |
| 2398102H1 | | 647 |
| 2531082H1 | | 698 |
| 2630745F6 | | 651 |
| 264639H1 | | 329 |
| 2704982H1 | | 313 |
| 2716787H1 | | 700 |
| 2798810F6 | | 835 |
| 2832401H1 | | 655 |
| 2894096F6 | | 837 |
| 2919406F6 | | 702 |
| 2937644F6 | | 838 |
| 2950021H1 | | 678 |
| 3010621F6 | | 679 |
| 3123948H1 | | 841 |
| 3253054R6 | | 843 |
| 3290073H1 | | 685 |
| 3330472H1 | | 844 |
| 335737H1 | | 362 |
| 3674358H1 | | 688 |
| 3749346F6 | | 848 |
| 3820429H1 | | 690 |
| 3978404F6 | | 849 |
| 4031124H1 | | 707 |
| 4056384H1 | | 708 |
| 4097060H1 | | 853 |
| 4288779H1 | | 726 |
| 4301823H1 | | 727 |
| 4558488F6 | | 712 |
| 4570377H1 | | 729 |
| 5058893F9 | | 733 |
| 5541621H1 | | |
| 5546249F6 | | 740 |
| 5546336H1 | | 860 |
| 5771839H1 | | 862 |
| 5804485H1 | | 863 |
| 5849807H1 | | 371 |
| 6530555H1 | | 866 |
| 656258H1 | | 372 |
| 6591535H1 | | 745 |
| 859993H1 | | 310 |
| 930273R6 | | 743 |
| 938765H1 | | 379 |

TABLE 3
Genes (By Genbank Accession Number)
modulated at least 1.5 fold in all cell lines and
at least 1.7 fold in patient responder sample.

| Gene ID | Accession No. | Seq ID |
|---|---|---|
| 5543360F8 | AC009220 | 738 |
| AA237071 | AL035420 | 467 |
| AA455969 | D00015 | 20 |
| M25315 | D12592 | 90 |
| U41078 | D26512 | |
| L10413 | L00634 | 164 |
| AA464627 | L09604 | 18 |
| 2470939H1 | L35848 | 830 |
| M84526 | M84526 | 88 |
| AI921879 | NM_002287 | 574 |
| AF204944 | NM_012105 | 527 |
| W77977 | NM_022336 | 815 |
| AA449040 | NM_024116 | 483 |
| AA521213 | S77359 | 473 |
| AA984230 | S80071 | 524 |
| AA456616 | U14970 | 22 |
| AI884916 | U29171 | 591 |
| U60519 | U60519 | 62 |
| X00351 | X13839 | 251 |
| AA868186 | X17093 | 425 |
| H27379 | X57198 | 144 |
| AA454585 | X75755 | 2 |
| X89576 | X89576 | 51 |
| AI580830 | XM_042041 | 562 |
| U49083 | Z49148 | 455 |
| 2398102H1 | | 647 |
| 2531082H1 | | 698 |

I claim:

1. A method of determining whether a patient with acute myelogenous leukemia will respond to treatment with an FTI by (a) analyzing a diseased cell from a bone marrow sample from the patient for a detectable difference in the amount of expression of a gene comprising Seq. ID. No. 846 (343105F7) following treatment with an FTI relative to an untreated diseased cell; (b) comparing the detectable difference from step (a) to those obtained from responder and non-responder patients; and (c) correlating the patient expression patter with that of a responder or non-responder to said FTI.

2. The method of claim 1 wherein the diseased cells are hematopoietic blast cells.

3. The method of claim 1 wherein the analysis step (a) is carried out using a nucleic acid array.

4. The method of claim 3 wherein the detectable difference is at least 1.5 fold compared to the expression of said genes in said non-responder.

5. The method of claim 3 wherein the detectable difference is at least 1.7 fold compared to the expression of said genes in said non-responder.

6. The method of claim 3 wherein the detectable difference is at least 2 fold compared to the expression of said genes in said non-responder.

7. The method of claim 1 wherein the FTI is selected from the group consisting of 7-(3-chlorophenyl)-9-[(4-chlorophenyl)-1H-imidazol-1-ylmeth-yl]-2,3-dihydro-1H,5H-benzo[ij]quinolizin-5-one, 7-(3- chlorophenyl)-9-[(4-chlorophenyl)-1H-imidazol-1-ylmethyl]-1,2-dihydro-4H-pyrrolo[3,2,1-ij]quin-oline-4-one, 8-[amino(4-chlorophenyl)(1-methyl-1H-imidazol-5-yl)methyl]-6-(3-chlorophen yl)-1,2-dihydro-4H-pyrrolo[3,2,1-ij]quinolin-4-one, 8-[amino(4-chlorophenyl)(1-methyl-1H-imidazol-5-yl)methyl]-6-(3-chlorophe-nyl)-2,3-dihydro-1H,5H-benzo[ij]quinolizin-5-one, and (B)-6-[amino(4-chlorophenyl)(1-methyl-1H-imidazol-5-yl)methyl]-4-(3-chlor-ophenyl)-1-methyl-2(1H)-quinolinone).

8. The method of claim 7 wherein the FTI is (B)-6-[amino(4-chlorophenyl)(1-methyl-1H-imidazol-5-yl)methyl]-4-(3-chlorophenyl)-1-methyl-2(1H)-quinoli-none).

9. A method of monitoring treatment response in a patient with acute myelogenous leukemia will respond to treatment with an FTI by (a) analyzing a diseased cell from a sample from the patient for a detectable difference in the amount of expression of a gene comprising Seq. ID. No. 846 (343105F7) at various periods throughout the course of treatment with said FTI; (b) comparing the expression pattern of step (a) to those obtained from responder and non-responder patients; and (c) correlating the patient expression patter with that of a responder or non-responder to said FTI to determine whether to adjust the treatment of the patient.

10. The method of claim 9 wherein the diseased cells are hematopoietic blast cells.

11. The method of claim 9 wherein the analysis step (a) is carried out using a nucleic acid array.

12. The method of claim 9 wherein the detectable difference is at least 1.5 fold compared to the expression of said genes in said non-responder.

13. The method of claim 9 wherein the detectable difference is at least 1.7 fold compared to the expression of said genes in said non-responder.

14. The method of claim 9 wherein the detectable difference is at least 2 fold compared to the expression of said genes in said non-responder.

15. The method of claim 9 wherein the FTI is selected from the group consisting of 7-(3-chlorophenyl)-9-[(4-chlorophenyl)-1H-imidazol-1-ylmeth-yl]-2,3-dihydro-1H,5H-benzo[ij]quinolizin-5-one, 7-(3-chlorophenyl)-9-[(4-chlorophenyl)-1H-imidazol-1-ylmethyl]-1,2-dihydro-4H-pyrrolo[3,2,1-ij]quin-oline-4-one, 8-[amino(4-chlorophenyl)(1-methyl-1H-imidazol-5-yl)methyl]-6-(3-chlorophen yl)-1,2-dihydro-4H-pyrrolo[3,2,1-ij]quinolin-4-one, 8-[amino(4-chlorophenyl)(1-methyl-1H-imidazol-5-yl)methyl]-6-(3-chlorophe-nyl)-2,3-dihydro-1H,5H-benzo[ij]quinolizin-5-one, and (B)-6-[amino(4-chlorophenyl)(1-methyl-1H-imidazol-5-yl)methyl]-4-(3-chlor-ophenyl)-1-methyl-2(1H)-quinolinone).

16. The method of claim 15 wherein the FTI is (B)-6-[amino(4-chlorophenyl)(1-methyl-1H-imidazol-5-yl)methyl]-4-(3-chlorophenyl)-1-methyl-2(1H)-quinoli-none).
